(19) United States
(12) Patent Application Publication
(10) Pub. No.: US 2017/0150951 A1
(43) Pub. Date: Jun. 1, 2017

(54) PROTECTION DEVICE FOR PREVENTING TUMOR DISSEMINATION AND TUMOR METASTASIS IN LAPAROSCOPIC SURGERY

(71) Applicants: Bin Ling, Beijing (CN); Andong Ling, Beijing (CN)

(72) Inventors: Bin Ling, Beijing (CN); Andong Ling, Beijing (CN)

(21) Appl. No.: 15/300,276

(22) PCT Filed: Jun. 25, 2014
(86) PCT No.: PCT/CN2014/000619

§ 371 Date: Sep. 28, 2016

(30) Foreign Application Priority Data
Mar. 28, 2014 (CN) 201410121274.6

(51) Int. Cl. A61B 17/00

(52) U.S. Cl. CPC A61B 17/00234; A61B 2017/00287

(57) ABSTRACT

A protection device for preventing tumor dissemination and tumor metastasis in a laparoscopic surgery includes a soft bag (1) for containing a resected tumor during the surgery provided in an abdominal cavity through a puncture in an abdominal wall, and a sealing thread (2) provided around a mouth of the soft bag (1). An instrument is able to be put into the soft bag in the abdominal cavity through the mouth of the soft bag (1) outside the abdominal cavity for inflating and crushing, or the instrument is able to be put into the soft bag in the abdominal cavity for operation and observation through a conduit shaped structure for a breaking operation (3) or a conduit shaped structure for an auxiliary operation (4) or a conduit shaped structure for peering (5).

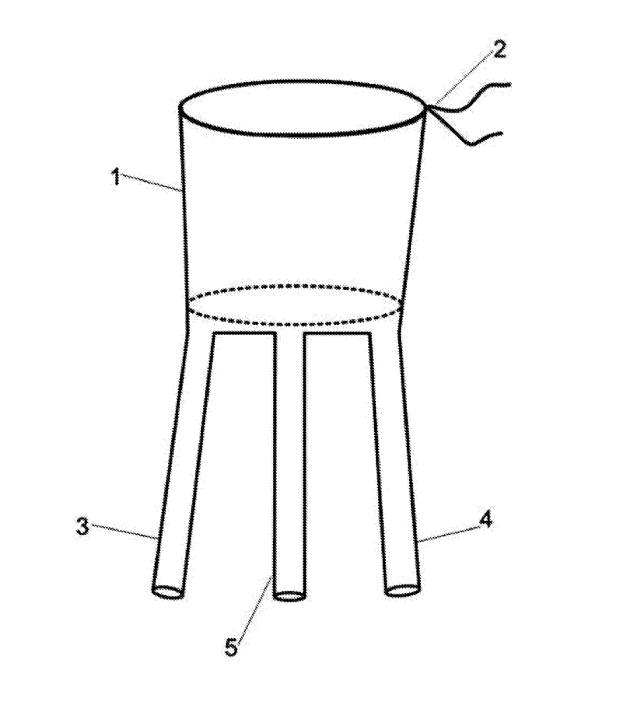

### CROSS REFERENCE OF RELATED APPLICATION

This is a U.S. National Stage under 35 U.S.C 371 of the International Application PCT/CN2014/000619, filed Jun. 25, 2014, which claims priority under 35 U.S.C. 119(a-d) to CN 201410121274.6, filed Mar. 28, 2014.

### BACKGROUND OF THE PRESENT INVENTION

Field of Invention

The present invention relates to a surgery auxiliary device, and more specifically to a protection device for preventing tumor dissemination and tumor metastasis in a laparoscopic surgery.

Description of Related Arts

The laparoscope surgery is characterized in that an incision in the abdominal wall is small and hurt and pain from a large incision in the abdominal wall in a traditional surgery are changed, so that it is known as “keyhole” surgery. The volume of tumor resected in the pelvic cavity or the abdominal cavity in the surgery is often large and it is difficult to take out the tumor through a puncture in the abdominal cavity, and in clinical practice, the tumor is needed to be broken by a mastax in the abdominal cavity to be taken out. However, tumor debris generated in the breaking process is able to easily cause the tumor to disseminate and implant in the pelvic cavity or the abdominal cavity, seriously threatening a patient's life.

### SUMMARY OF THE PRESENT INVENTION

The invention provides a protection device for preventing tumor dissemination and tumor metastasis in a laparoscopic surgery in order to avoid the existing shortcomings in the prior art as above, wherein tumor tissues resected in the abdominal cavity are isolated in the closed cavity to be broken, and tumor debris generated in the breaking process are confined in the closed cavity to avoid dissemination and metastasis.

The invention adopts the following technical solutions to solve the technical problem:

A protection device for preventing tumor dissemination and tumor metastasis in a laparoscopic surgery, comprises a soft bag for containing a resected tumor during the surgery provided in an abdominal cavity through a puncture in an abdominal wall, and a sealing thread provided around a mouth of the soft bag, wherein: the sealing thread is tightened and ligated so as to enable the mouth of the soft bag to be closed, the closed mouth of the soft bag is able to be stretched out of the abdominal cavity through the puncture in the abdominal wall via the sealing thread, and an instrument is able to be put into the soft bag in the abdominal cavity through the mouth of the soft bag outside the abdominal cavity, a relatively closed larger space is formed in the closed abdominal cavity through inflating the soft bag via a one-way gas valve, thereby directly completing processes of crushing and taking out tissue samples.

Preferably, in the protection device for preventing tumor dissemination and tumor metastasis in the laparoscopic surgery, a conduit communicated with the soft bag is provided at a tail area of the soft bag, the conduit is able to be a single or multiple, an external port of the conduit is able to be maintained outside the abdominal cavity or stretched out of the abdominal cavity through the puncture(s) in the abdominal wall, and the instrument is able to be put into the soft bag in the abdominal cavity through the external port of the conduit.

Preferably, in the protection device for preventing tumor dissemination and tumor metastasis in the laparoscopic surgery, the external port of the conduit is able to be closed and the soft bag is aerated through inflating the soft bag via the external port of the conduit.

Preferably, in the protection device for preventing tumor dissemination and tumor metastasis in the laparoscopic surgery, the external port of the conduit is able to be positioned into an abdominal wall puncture outfit with a one-way gas valve, the instrument is supported by the abdominal wall puncture outfit; and the one-way gas valve is the one-way gas valve opened towards the soft bag in one direction.

Preferably, in the protection device for preventing tumor dissemination and tumor metastasis in the laparoscopic surgery, the soft bag is a transparent body, and the instrument which passes through conduit comprises a grasper and a mastax.

Preferably, in the protection device for preventing tumor dissemination and tumor metastasis in the laparoscopic surgery, the soft bag is an opaque body, and the instrument which passes through conduit comprises a grasper, a mastax and a laparoscope.

Compared to the prior arts, the invention has the advantages as follows.

1. The invention provides a protective measure for preventing tumor dissemination and tumor metastasis based on conventional methods for breaking a tumor adopted in the laparoscopic surgery.

2. The application of the invention enables the laparoscope to be put into the soft bag through the conduit to directly observe the tumor breaking process, avoiding broken cutters to injury normal tissues.

3. The application of the invention also enables the laparoscope to observe the tumor breaking process through the transparent soft bag, avoiding broken cutters to injury normal tissues.

4. The application of the invention aerates the soft bag through the conduit in the existing closed inflated abdominal cavity in the laparoscope surgery to form another relatively closed space, greatly facilitating the breaking operation of the tumor tissues.

### BRIEF DESCRIPTION OF THE DRAWINGS

The drawing is a structurally schematic view of a protection device for preventing tumor dissemination and tumor metastasis in a laparoscopic surgery according to the invention.

Reference numbers, 1: soft bag; 2: sealing thread; 3: conduit shaped structure for breaking operation; 4: conduit shaped structure for auxiliary operation; 5: conduit shaped structure for peering.

### DETAILED DESCRIPTION OF THE PREFERRED EMBODIMENT

Referring to the drawing, a protection device for preventing tumor dissemination and tumor metastasis in a laparoscopic surgery according to the present embodiment comprises a soft bag 1 for containing a resected tumor during the surgery which is provided in an abdominal cavity through a puncture in a abdominal wall, and a sealing thread 2 provided around a mouth of the soft bag 1, wherein: the sealing thread 2 is tightened and ligated after a tumor sample is put into the soft bag 1 so as to enable the mouth of the soft bag 1 to be closed in a pelvic cavity or the abdominal cavity; or the closed mouth of the soft bag 1 is able to be stretched out of the abdominal cavity through the puncture in the abdominal wall via the sealing thread 2, and an instrument is able to be put into the soft bag in the abdominal cavity through the mouth of the soft bag 1 outside the abdominal cavity.

In specific implementation, a conduit communicated with the soft bag is provided at a tail area of the soft bag 1, and an external port of the conduit is able to be maintained outside the abdominal cavity or stretched out of the abdominal cavity through other puncture in the abdominal wall, and the instrument is able to be put into the soft bag in the abdominal cavity through the external port of the conduit. An abdominal wall puncture outfit with a one-way gas valve is provided in the external port of the conduit and the soft bag 1 is aerated through inflating the soft bag 1 via the external port of the conduit. The instrument is supported by the abdominal wall puncture outfit; and the one-way gas valve is the one-way gas valve opened towards the soft bag in one direction.

In specific implementation, the soft bag 1 is able to be a transparent body, and the instrument positioned in the soft bag comprises a grasper and a mastax. For this structure, a laparoscope is positioned outside the transparent body to observe an operation. The soft bag 1 is also able to be an opaque body, and the instrument comprises a grasper, a mastax and a laparoscope. For this structure, the laparoscope is positioned within the opaque body to observe the operation.

Operation Process:

The soft bag is placed into the abdominal cavity through the puncture in the abdominal wall after fold or roll-up, the resected tumor tissue sample is put into the soft bag, the sealing thread is stretched to close the mouth; or the sealing thread is sequentially stretched to enable the mouth of the soft bag to be pulled out of the abdominal cavity through the puncture in the abdominal wall, the instrument is put into the soft bag through the mouth of the soft bag to carry out the breaking operation, and the mouth-closed soft bag is directly pulled out after the breaking operation.

An external port of the conduit is maintained outside the abdominal cavity or stretched out of the abdominal cavity through punctures 3, 4 and 5, and the laparoscope or other instrument is put into the soft bag through the conduit for inflation, observation and operation.

In specific implementation, the soft bag is aerated to be sufficiency expanded in the abdominal cavity, that is, another relatively closed independent space is constructed in the existing closed inflated abdominal cavity, the laparoscope is able to be directly put into the independent space through the conduit for observation or be positioned outside the transparent soft bag for observation, the entire process for breaking tumor is completed in the closed independent space, the broken tumor tissues are taken out in the independent space one by one, and the tumor debris tissues generated during the process are enclosed in the independent space.

1. A protection device for preventing tumor dissemination and tumor metastasis in a laparoscopic surgery, comprising a soft bag (1) for containing a resected tumor during the surgery provided in an abdominal cavity through a puncture in an abdominal wall, and a sealing thread (2) provided around a mouth of the soft bag (1), wherein: the sealing thread (2) is tightened and ligated so that the mouth of the soft bag (1) is closed in a pelvic cavity or the abdominal cavity; or the closed mouth of the soft bag (1) is able to be stretched out of the abdominal cavity through the puncture in the abdominal wall via the sealing thread (2), and an instrument is able to be put into the soft bag in the abdominal cavity through the mouth of the soft bag (1) outside the abdominal cavity, a relatively closed larger space is formed in the closed abdominal cavity through inflating the soft bag via a one-way gas valve, thereby directly completing processes of crushing and taking out tissue samples.

2. The protection device for preventing tumor dissemination and tumor metastasis in the laparoscopic surgery according to claim 1, wherein a conduit communicated with the soft bag is provided at a tail area of the soft bag (1), the conduit is able to be one (3) or multiple (4 or 5), an external port of the conduit is able to be maintained outside the abdominal cavity through a single or multiple punctures in the abdominal wall, and a laparoscope or other instrument is able to be put into the soft bag (1) in the abdominal cavity through the external port of the conduit.

3. The protection device for preventing tumor dissemination and tumor metastasis in the laparoscopic surgery according to claim 2, wherein the external port of the conduit is able to be closed and the soft bag (1) is aerated through inflating the soft bag (1) via the external port of the conduit.

4. The protection device for preventing tumor dissemination and tumor metastasis in the laparoscopic surgery according to claim 3, wherein the external port of the conduit is able to be positioned into an abdominal wall puncture outfit with an one-way gas valve, the instrument is supported by the abdominal wall puncture outfit; and the one-way gas valve is the one-way gas valve opened towards the soft bag in one direction.

5. The protection device for preventing tumor dissemination and tumor metastasis in the laparoscopic surgery according to claim 1, wherein the soft bag (1) is a transparent body, and the instrument comprises a grasper and a mastax.

6. (canceled)

7. The protection device for preventing tumor dissemination and tumor metastasis in the laparoscopic surgery according to claim 2, wherein the soft bag (1) is a transparent body, and the instrument which passes through the conduit comprises a grasper and a mastax.

8. The protection device for preventing tumor dissemination and tumor metastasis in the laparoscopic surgery according to claim 3, wherein the soft bag (1) is a transparent body, and the instrument which passes through the conduit comprises a grasper and a mastax.

9. The protection device for preventing tumor dissemination and tumor metastasis in the laparoscopic surgery according to claim 4, wherein the soft bag (1) is a transparent body, and the instrument which passes through the conduit comprises a grasper and a mastax.

10. The protection device for preventing tumor dissemination and tumor metastasis in the laparoscopic surgery according to claim 1, wherein the soft bag (1) is an opaque body, and the instrument comprises a grasper, a mastax and a laparoscope.

11. The protection device for preventing tumor dissemination and tumor metastasis in the laparoscopic surgery according to claim 2, wherein the soft bag (1) is an opaque body, and the instrument which passes through the conduit comprises a grasper, a mastax and a laparoscope.

12. The protection device for preventing tumor dissemination and tumor metastasis in the laparoscopic surgery according to claim 3, wherein the soft bag (1) is an opaque body, and the instrument which passes through the conduit comprises a grasper, a mastax and a laparoscope.

13. The protection device for preventing tumor dissemination and tumor metastasis in the laparoscopic surgery according to claim 4, wherein the soft bag (1) is an opaque body, and the instrument which passes through the conduit comprises a grasper, a mastax and a laparoscope.
